(19) United States
(12) Patent Application Publication
(10) Pub. No.: US 2010/0197826 A1
(43) Pub. Date: Aug. 5, 2010

(54) Wound Closing Compounds With Additives

(76) Inventors: Rajat Agrawal, Redlands, CA (US); Dean Eliott, Los Angeles, CA (US); Mark Humayun, Glendale, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/363,594

(22) Filed: Jan. 30, 2009

(51) Int. Cl. A61K 47/34; C08G 69/00

(52) U.S. Cl. 523/118; 528/271

(57) ABSTRACT

According to one embodiment of the present invention, there is provided a polyacrylamide or derivative thereof, such as poly-N-isopropyl acrylamide (pNIPAM) for use in the closure of wounds. Also provided is a composition comprising a polyacrylamide or derivative thereof in an amount effective for closing a wound, at least in part, and one or more pharmaceutically acceptable carriers. The a polyacrylamide or derivative thereof may be used in the composition according to the invention in various concentrations and may be combined with other therapeutic compounds. In another embodiment, a method for treating a wound with the compounds and compositions described herein is provided. The method comprises administering to a mammal a polyacrylamide or derivative thereof in an amount effective to close at least a part of a wound.

### CROSS-REFERENCE TO RELATED APPLICATIONS

This application claims the benefit of International Application No. PCT/US09/32700 filed Jan. 30, 2009, and U.S. Provisional Patent Application No. 61/024,643, filed Jan. 30, 2008, the contents of both of which are incorporated herein by reference in their entirety.

### BACKGROUND

A wound is an internal or external bodily injury or lesion caused by mechanical, chemical, viral, bacterial, fungal and other pathogenic organisms, or thermal means, which disrupt the normal continuity of tissue structure. Wounds may be caused by accident, pathological organisms, or by surgical procedures. Sutures or other forms of closure devices, e.g., staples, and glues have long been used to close surgical wounds and lacerations. These forms of wound closure help in closing wounds, but the use and placement of sutures is time consuming and can lead to side-effects such as infection around the suture and delay in healing.

Wounds resulting from traumatic loss of tissue may be particularly difficult to close, due to the difficult apposition of the two edges. Irregular edges of a wound may cause inadequate wound edge apposition that does not allow for a water-tight closure. If a suture apposition is not adequate, it can lead to serious consequences such as leakage or infection. Further, there are known side-effects to the use of sutures, including increased chance of infection, in and around the suture site, or delayed healing, and in the case of ocular wounds, change in refractive status.

Sutures for corneal or scleral wounds are known to be problematic. Corneal sutures may cause changes in the refractive power of the eye, which may lead to visual problems for the patient. Also, ocular hypotony may lead to development of serious side-effects like retinal and choroidal detachment or endophthalmitis. These complications can sometimes lead to serious ocular consequences including but not limited to total blindness. Also, placing sutures in the eye adds to the operating room time, which increases the cost of medical care.

Biological glues used to close wounds are known. For example, cyanoacrylate glue has been used in the closure of corneal wounds. However, known biological glues have not been found to be very effective, or suffer from other disadvantages. Some known biological glues require a dry surface for application, which may not be possible in some circumstances, e.g., around the eye, and some known biological glues have been found to be toxic to biological tissues.

Accordingly, a biological glue for closing wounds that prevents the side effect of the current technology is needed.

### SUMMARY

According to one embodiment of the invention, a compound for use in the closure of wounds that satisfies the above-identified needs is provided. The compound comprises a polyacrylamide or a derivative thereof.

According to another embodiment of the invention, a composition for use in the closure of a wound is provided. The composition comprises a polyacrylamide or a derivative thereof and one or more pharmaceutically acceptable carriers.

According to another embodiment of the invention, a composition for use in the closure of a wound is provided. The composition comprises a polyacrylamide or a derivative thereof and one or more therapeutic compounds. Optionally, one or more pharmaceutically acceptable carriers is added to the composition. Preferably, the therapeutic compound is selected from the group consisting of antibiotics, antifungal agents, growth factors, inhibitory growth factors, steroids, and combinations thereof.

According to another embodiment of the invention, a method for treating a wound is provided. The method comprises the administration to a mammal of a polyacrylamide or a derivative thereof in an amount effective to close at least a part of a wound. According to another embodiment of the method, the method comprises the administration to the mammal a composition comprising a polyacrylamide or a derivative thereof and one or more therapeutic compounds, and/or one or more pharmaceutically acceptable carriers.

### DESCRIPTION

According to the present invention, there is provided a compound, a polyacrylamide or a derivative thereof for use in the closure of wounds that satisfies the above-identified needs. In general, the polyacrylamide or derivative thereof has an adhesive property.

In a preferred embodiment, the polyacrylamide or derivative thereof changes from a hydrophilic to a hydrophobic state with a change in temperature, and/or has high mechanical strength, and/or is transparent.

The polyacrylamides are compounds of Formula I:

(—CH₂CH(CONH₂)—)ₙ I

Derivatives of the polyacrylamide include, but are not limited to one or more N-substitutions on the amide moiety, and/or substitutions on one or more carbons of the ethylene backbone of the polyacrylamide polymer. Preferably the polyacrylamide is N-substituted with one or more alkyl, aryl, arylalkylene, alkylaryl, cycloalkyl, cycloalkylalkylene, alkylcycloalkyl, heterocyclyl, alkylheterocyclyl, heterocyclylalkylene, heteroaryl, heteroarylalkylene, and alkylheteroaryl, or the N forms a nitrocyclic ring with the substituents to which it is attached.

According to a preferred embodiment of the invention, the polyacrylamide or derivative thereof is a compound of Formula II with an adhesive property.

(—C(R₁)(R₂)CR₃(CONR₄R₅)—)ₙ II

Wherein,

R₁, R₂, and R₃ are each independently selected from the group consisting of hydrogen, halogen, C₁-C₆ alkyl, C₁-C₆ aryl, and C₁-C₆ cycloalkyl,

R₄ and R₅ are each independently selected from the group consisting of hydrogen, C₁-C₁₀ alkyl, C₁-C₁₀ aryl, C₁-C₁₀ arylalkylene, C₁-C₁₀ alkylaryl, C₁-C₁₀ cycloalkyl, C₁-C₁₀ cycloalkylalkylene, C₁-C₁₀ alkylcycloalkyl, C₁-C₁₀ heterocyclyl, C₁-C₁₀ alkylheterocyclyl, C₁-C₁₀ heterocyclylalkylene, C₁-C₁₀ heteroaryl groups, C₁-C₁₀ heteroarylalkylene, and C₁-C₁₀ alkylheteroaryl, or R₄ and R₅ are taken together with the N to which they are attached to form a five- or six-membered nitrocyclic ring; and

n is an integer which provides a molecular weight of the polyacrylamide of between about 2×10³ to about 3×10⁵.

In a preferred embodiment, the polyacrylamide compound is poly-N-isopropylacrylamide (“pNIPAM”, as used herein, and variously abbreviated, PNIPAM, PNIPA, PNIPAA). Poly-N-isopropylacrylamide has been found to be biocompatible with human tissue, including the eye. Since poly-N-isopropylacrylamide is biocompatible, it will not cause any damage to the human tissues and can be used without fear of it accidentally entering the body cavities, e.g. the anterior or posterior segment of the eye, during or after application. Further, poly-N-isopropylacrylamide has unique chemical properties, in that it changes from hydrophilic to hydrophobic state with a change in temperature. This hydrophilic to hydrophobic change gives poly-N-isopropylacrylamide adhesive properties that may be utilized in closure of wounds. Poly-N-isopropylacrylamide also has high mechanical strength and is transparent.

The use of a polyacrylamide with adhesive properties, such as poly-N-isopropylacrylamide to close wounds in mammals is applicable to any kind of wound closure in the body, including planned surgical and/or accidental wounds including trauma. The technology described herein may be utilized in closure of wounds, including but not limited to ocular wounds like sclerotomies that are made for surgical intervention. Also, in cases of small incision surgery for any part of the human body (e.g., 23 or 25-gauge in the eye), where leakage can occasionally be an issue, the glue can be an additional source of closure. Traumatic wounds in the body lead to occasional loss of tissue leading to difficulty in adequate wound apposition. This can be a serious issue including, but not limited to ocular trauma. Corneal lacerations present to the emergency room frequently. Due to loss of tissue during trauma, there is great difficulty in apposing the wounds. This leads to extensive scarring and subsequent refractive changes. The use poly-N-isopropylacrylamide to close wounds may help better manage these patients.

The use of a polyacrylamide with adhesive properties, such as poly-N-isopropylacrylamide for closure of body wounds, including sclerotomies and other ocular wounds may prevent the side effects as seen by use of the current technology. It may be possible to close extensive surgical wounds with precise apposition of the two edges of the wound, and thus preventing complications including but not limited to wound leakage and infection. In ocular wounds, it is likely to prevent refractive changes, which cause visual problems to the patient after wound closure. Suturing wounds increases operating room time, which increases costs.

In another embodiment, a polyacrylamide with adhesive properties, such as poly-N-isopropylacrylamide is used to treat a large wound. Large wounds may require sutures for apposition of the two lips of the wound (i.e., abdominal wounds). But the extent of the wound, and its intentional or accidental construction may not allow a good closure. The use of poly-N-isopropylacrylamide may be an additional step to the closure of these wounds.

In another embodiment, the invention comprises a composition comprising a polyacrylamide with adhesive properties, such as poly-N-isopropyl acrylamide (pNIPAM) in an amount effective for closing a wound, at least in part, and one or more pharmaceutically acceptable carriers. In a preferred embodiment, the compositions of the invention are topical compositions, which are used to treat wounds such as incisions and lacerations. The compositions may be used externally to treat skin wounds, or may be used internally, to treat organ wounds. In a more preferred embodiment, the compositions may be used in ophthalmological preparations to treat surgically induced wounds and accidental wounds in the eye.

In another embodiment, the invention is a composition for use in the closure of a wound comprising a polyacrylamide with adhesive properties, such as poly-N-isopropylacrylamide, one or more therapeutic compound, such as a medication, and one or more pharmaceutically acceptable carriers. Incorporating medications into the compound may prevent repeated oral, injectable or topical use of medications.

A polyacrylamide with adhesive properties, such as poly-N-isopropylacrylamide may be used in the composition according to the invention in various concentrations and may be combined with other therapeutic compounds, such as a pharmaceutically active compound, i.e., a medication. Examples of therapeutic compounds include, but are not limited to, antibiotics, antifungal agents, growth factors, inhibitory growth factors, and steroids. The therapeutic compounds may be added to the composition for purposes of additional usage, or for other purposes, including but not limited to drug delivery. Other therapeutic compounds include therapeutic wound healing compounds, known to contribute to wound healing, such as vitamins and minerals, and other medicaments, which enable faster and easier healing.

In another embodiment, a therapeutic medication is given to a patient on a routine basis after surgery or trauma to prevent or treat infection. These are usually given either in oral tablet or capsule form, injected subcutaneously/intramuscularly, as eye drops or injected into the eye. Incorporating medications into the compositions of the invention may prevent repeat intake of medications by the patient, or avoid repeated injections into the eye or the body.

The types of wounds which may be closed using the compounds and compositions of the present invention are those which result from an injury which causes epidermal damage such as incisions, wounds in which the skin is broken by a cutting instrument, and lacerations, wounds by which the skin is broken by a blunt or dull instrument.

In another embodiment, the invention is a method for treating a wound with the compounds and compositions described herein. The method comprises administering to a mammal an amount of a polyacrylamide with adhesive properties, such as poly-N-isopropylacrylamide, in an amount effective to close at least a part of a wound.

In another embodiment, the invention is a method for treating a mammal having a wound, as described herein. The method comprises the administration to the mammal a pharmaceutical composition comprising a polyacrylamide with adhesive properties, such as poly-N-isopropylacrylamide, one or more therapeutic compounds, and one or more pharmaceutically acceptable carriers. Preferably, the polyacrylamide is applied to the wound when the temperature of the mammal, or at least the tissue surrounding the wound is greater than at least about 32 degrees centigrade.

Human body temperature is usually around 37 degrees centigrade. But, different parts of the body have different temperatures, which may be dependent on, but not limited to their exposure to the outside environment or the amount of blood supply. In situations of intentional low temperature environment, including but not limited to operating rooms, certain parts of the body show lower temperature.

According to one embodiment of method of the invention, frequent temperature checks are undertaken during the method of wound closing. This may be done by a thermostat or any other type of temperature reader, which may even be a sensor temporarily placed around the body part that is being considered for the use of the compound. If the temperature is below 31 degrees centigrade, controlled increase in temperature of that body part is undertaken. Methods for increasing the temperature of the part of the body can include, but are not limited to, warm medical grade saline solution {e.g. balanced salt solution (BSS)}. In another embodiment, a delivery instrument containing a polyacrylamide will increase the temperature of the compound as it leaves the instrument. Once the temperature exceeds 32 degrees centigrade a polyacrylamide is applied to any internal or external surface of the body. The application of the compound can be in the form of, but not limited to, liquid, gel or solid. After approximately 5 minutes, the body surface or part (i.e., tissue surrounding the wound) is allowed to cool, e.g. by stopping the method used for heating that body part. Sometimes additional use of cooling substances or liquids may be required.

According to another embodiment of method of the invention, frequent testing is undertaken to check whether the wound has sealed adequately. The wound can be visually inspected or tested by another means, including but not limited to use of dyes. Then, routine steps in surgery would follow thereafter.

The use of a polyacrylamide, specifically, poly-N-isopropylacrylamide, to close wounds in animals, including enucleated porcine as well as live rabbit eyes has been evaluated. These animals have been followed up for many months and have demonstrated consistent closure of the wounds. The outcomes have been tested with repeat examinations and intraocular pressure checks. Histopathology examination has shown good closure of these wounds. However, the wound closing technology is applicable to other mammals including humans.

Although the present invention has been discussed in considerable detail with reference to certain preferred embodiments, other embodiments are possible. Therefore, the scope of the appended claims should not be limited to the description of preferred embodiments contained herein.

1. A compound, for use in the closure of wounds, the compound comprising a polyacrylamide or a derivative thereof.

2. A compound according to claim 1 wherein the polyacrylamide or derivative thereof has an adhesive property.

3. A compound according to claim 1 wherein the polyacrylamide or derivative thereof changes from a hydrophilic to a hydrophobic state with a change in temperature.

4. A compound according to claim 1 wherein the polyacrylamide or derivative thereof is a compound of Formula II with an adhesive property:
(—C(R₁)(R₂)CR₃(CONR₄R₅)—)ₙ II
wherein
R₁, R₂, and R₃ are each independently selected from the group consisting of hydrogen, halogen, C₁-C₆ alkyl, C₁-C₆ aryl, and C₁-C₆ cycloalkyl,
R₄ and R₅ are each independently selected from the group consisting of hydrogen, C₁-C₁₀ alkyl, C₁-C₁₀ aryl, C₁-C₁₀ arylalkylene, C₁-C₁₀ alkylaryl, C₁-C₁₀ cycloalkyl, C₁-C₁₀ cycloalkylalkylene, C₁-C₁₀ alkylcycloalkyl, C₁-C₁₀ heterocyclyl, C₁-C₁₀ alkylheterocyclyl, C₁-C₁₀ heterocyclylalkylene, C₁-C₁₀ heteroaryl groups, C₁-C₁₀ heteroarylalkylene, and C₁-C₁₀ alkylheteroaryl, or R₄ and R₅ are taken together with the N to which they are attached to form a five- or six-membered nitrocyclic ring.

5. A compound according to claim 1 wherein the polyacrylamide derivative is N-substituted with at least one C₁-C₁₀ alkyl group.

6. A compound according to claim 1 wherein the polyacrylamide is poly-N-isopropylacrylamide or a derivative thereof.

7. A composition for use in the closure of a wound, the composition comprising:
a compound according to claim 1; and
one or more pharmaceutically acceptable carriers.

8. A composition according to claim 7 wherein the compound is poly-N-isopropylacrylamide or a derivative thereof.

9. A composition for use in the closure of a wound, the composition comprising:
a compound according to claim 1; and
one or more therapeutic compounds.

10. A composition according to claim 9 wherein the compound is poly-N-isopropylacrylamide or a derivative thereof.

11. A composition according to claim 9 wherein the therapeutic compound is selected from the group consisting of antibiotics, antifungal agents, growth factors, inhibitory growth factors, steroids, and combinations thereof.

12. A method for treating a wound, the method comprising the administration to a mammal of a compound comprising a polyacrylamide or a a derivative thereof in an amount effective to close at least a part of a wound.

13. The method according to claim 12 wherein the compound is poly-N-isopropylacrylamide or a derivative thereof.

14. A method for treating a mammal having a wound, the method comprising the administration to the mammal of a composition in amount effective to close at least a part of a wound, the composition comprising:
a compound comprising a polyacrylamide or a derivative thereof, and;
one or more therapeutic compounds.

15. The method of claim 14, wherein the compound is poly-N-isopropylacrylamide or a derivative thereof.

16. The method of claim 14, wherein the one or more therapeutic compounds is selected from the group consisting of antibiotics, antifungal agents, growth factors, inhibitory growth factors, steroids, and combinations thereof.

17. The method of claim 14, wherein the composition further comprises one or more pharmaceutically acceptable carriers.

18. The method of claim 12, wherein the polyacrylamide or derivative thereof changes from a hydrophilic state to a hydrophobic state with a change in temperature.

19. The method of claim 12, wherein the polyacrylamide or derivative thereof is a compound of Formula II with an adhesive property:
(—C(R₁)(R₂)CR₃(CONR₄R₅)—)ₙ II
wherein
R₁, R₂, and R₃ are each independently selected from the group consisting of hydrogen, halogen, C₁-C₆ alkyl, C₁-C₆ aryl, and C₁-C₆ cycloalkyl,
R₄ and R₅, are each independently selected from the group consisting of hydrogen, C₁-C₁₀ alkyl, C₁-C₁₀ aryl, C₁-C₁₀ arylalkylene, alkylaryl, C₁-C₁₀ cycloalkyl, C₁-C₁₀ cycloalkylalkylene, alkylcycloalkyl, C₁-C₁₀ heterocyclyl, C₁-C₁₀ alkylheterocyclyl, C₁-C₁₀ heterocyclylalkylene, C₁-C₁₀ heteroaryl groups, C₁-C₁₀ heteroarylalkylene, and C₁-C₁₀ alkylheteroaryl, or R₄, and R₅, are taken together with the N to which they are attached to form a five- or six-membered nitrocyclic ring.

20. The method of claim 12, wherein the polyacrylamide or derivative thereof is a polyacrylamide derivative that is N-substituted with at least one C₁-C₁₀ alkyl group.
